(19) United States
(12) Patent Application Publication
(10) Pub. No.: US 2005/0148546 A1
(43) Pub. Date: Jul. 7, 2005

(54) Deacetylation of N-acetylglucosamine

(76) Inventors: Alan D. Grund, Manitowoc, WI (US); Thomas A. Jerrell JR., Manitowoc, WI (US)

Correspondence Address: SHERIDAN ROSS PC, 1560 BROADWAY, SUITE 1200, DENVER, CO, 80202, US

(21) Appl. No.: 11/009,376

(22) Filed: Dec. 9, 2004

Related U.S. Application Data
(60) Provisional application No. 60/528,457, filed on Dec. 9, 2003.

(51) Int. Cl. A61K 31/7008; C07H 5/04

(52) U.S. Cl. 514/62; 536/55.2

(57) ABSTRACT

The invention provides methods of deacetylating N-acetylglucosamine by solid acid hydrolysis on a cation exchange resin. These methods advantageously reduce the amount of acid used and the need for acid recycle facilities. The isolated glucosamine salt produced is very clean, reducing or eliminating the need for further purification steps to decolorize or desalt the product. Using these methods, the number of crystallization steps required are minimized and the degradation of the N-acetylglucosamine in solution is significantly reduced leading to increased yield of high quality glucosamine.

### CROSS REFERENCE TO RELATED APPLICATION

This application claims the benefit of priority under 35 U.S.C. § 119(e) to U.S. Provisional Patent Application No. 60/528,457, filed Dec. 9, 2003, which is incorporated herein in its entirety by this reference.

### FIELD OF THE INVENTION

The invention resides in the field of solid acid hydrolysis of amino sugars.

### BACKGROUND OF THE INVENTION

Glucosamine is an amino derivative of glucose and is an important constituent of many natural polysaccharides that can form structural materials for cells, analogous to structural proteins. Glucosamine is manufactured as a nutraceutical product for the treatment of osteoarthritic conditions in animals and humans.

Glucosamine is obtained predominately by acid hydrolysis of chitin, a complex carbohydrate derived from N-acetyl-D-glucosamine. Alternatively, glucosamine can also be produced by acid hydrolysis of variously acetylated chitosans. These processes suffer from poor product yields (in the range of 50% conversion of substrate to glucosamine). Moreover, the availability of raw material (i.e., a source of chitin, such as crab shells) is becoming increasingly limited. Glucosamine has also been obtained by the hydrolysis of N-acetylglucosamine (NAG) to glucosamine by reacting NAG with an acid. While this represents an improvement in the production of glucosamine, it requires large amounts of acid and consequently, acid recycle facilities. Additionally, the glucosamine solution produced by this method must often be decolorized before the pH is adjusted and the solution is washed and concentrated. These steps reduce the yield to levels lower than desired and drive up the cost of the glucosamine on a per weight basis. Therefore, there is a need in the industry for a more cost-effective method for producing high yields of glucosamine for commercial sale and use.

### SUMMARY OF THE INVENTION

The present invention is directed to a method of deacetylating N-acetylglucosamine by contacting N-acetylglucosamine with a cation exchange resin. In this process, an acetyl group is hydrolyzed to produce glucosamine. In the process, glucosamine can become bound to the cation exchange resin, and the cation exchange resin can be in the hydrogen ion form. The step of contacting can be performed at a pH range of about 2 to about 5. The step of contacting can be performed at a temperature of between about 20° C. and about 150° C.; between about 100° C. and about 115° C., or at about 110° C. The step of contacting can be performed under a pressure of between about 3 pounds per square inch (psig) and about 50 psig although it should be recognized that the pressure required will depend upon the vapor pressure produced by bringing the aqueous reactants to the desired reaction temperature. The molar ratio of resin functional groups to N-acetylglucosamine can be between about 1:1 to about 5:1; or between about 2:1 and about 3:1. The contacting step can include drawing off water vapor and acetic acid from a reactor containing the cation exchange resin.

In another embodiment, the invention can also include eluting glucosamine from the cation exchange resin, which can be washing the cation exchange resin with at least two water washes; or washing the cation exchange resin with a wash solution containing one or more cations, preferably high-activity cations. The step of eluting can also be washing the cation exchange resin with a wash solution containing an acid, which can be hydrochloric acid or sulfuric acid. In another embodiment, the process can include washing glucosamine bound to the cation exchange resin. In another embodiment, the process can include crystallizing glucosamine after elution from the cation exchange resin.

In one embodiment, the N-acetylglucosamine is in a solution at a concentration of between about 10% and about 70%, or between about 20% and about 50%, prior to contact with the cation exchange resin. The N-acetylglucosamine can be contacted with the cation exchange resin for a period of between about 30 minutes and about 4 hours. Also, the N-acetylglucosamine can be contacted with the cation exchange resin in the presence of a gas, such as nitrogen gas.

In a further embodiment, the N-acetylglucosamine is produced by a fermentation process, and the N-acetylglucosamine can be present in a fermentation medium. In this embodiment, the process can include precipitating N-acetylglucosamine-containing solids from the fermentation medium prior to contacting N-acetylglucosamine with a cation exchange resin. The process can also include crystallizing N-acetylglucosamine-containing solids from the fermentation medium prior to contacting N-acetylglucosamine with a cation exchange resin. In this embodiment, the fermentation medium can be partially purified, e.g., to remove cellular material, biomass, cations and anions.

A further embodiment of the present invention is a method of producing glucosamine that includes contacting N-acetylglucosamine with a cation exchange resin wherein the acetyl group is hydrolyzed to produce glucosamine; washing the cation exchange resin with at least one water wash; and, washing the cation exchange resin with an acid to elute glucosamine.

A further embodiment of the present invention is a method of producing glucosamine that includes culturing a microorganism in a fermentation medium to produce N-acetylglucosamine and deionizing the fermentation medium. This process also includes contacting the N-acetylglucosamine with a cation exchange resin to hydrolyze the acetyl group to produce glucosamine. The cation exchange resin is washed with at least one water wash and washed with hydrochloric acid to elute glucosamine. The eluted glucosamine is contacted with an aqueous alcohol to crystallize the glucosamine, and the glucosamine crystals are dried.

### BRIEF DESCRIPTION OF THE DRAWINGS

FIG. 1 shows a time-course of NAG hydrolysis according to a method of the present invention using DOW 650C™ resin at 100° C.

FIG. 2 shows a time-course of NAG hydrolysis according to a method of the present invention using AMBERLYST 119™ resin at 100° C.

FIG. 3 shows a time-course of NAG hydrolysis according to a method of the present invention using AMBERLYST 39™ resin at 100° C.

FIG. 4 shows a time-course of NAG hydrolysis according to a method of the present invention using AMBERLYST 119™ resin at 100° C.

FIG. 5 shows a time-course of NAG hydrolysis according to a method of the present invention using a DOW M31™ resin at 90° C.

FIG. 6 shows concentrations of N-acetylglucosamine and acetate, and the profile of a water wash in a time-course during the hydrolysis of N-acetylglucosamine according to the method of the present invention using a DOW 650C™ column at 90° C.

FIG. 7 shows the physical and chemical parameters of the column hydrolysis reaction depicted in FIG. 6.

FIG. 8 shows concentrations of N-acetylglucosamine and acetate, as well as pH and conductivity and the profile of a water wash during the hydrolysis of N-acetylglucosamine using a DOW 650C™ column.

FIG. 9 shows the time-course for N-acetylglucosamine disappearance and acetate formation in a DOW 650C™ column.

FIG. 10 shows N-acetylglucosamine hydrolysis with DOW MONOSPHERE 88™ resin in a FPCL column.

FIG. 11 shows N-acetylglucosamine hydrolysis using DOW M-31™ resin.

FIG. 12 shows a comparison of N-acetylglucosamine hydrolysis reactions performed according the methods of the present invention using two different resins and conducted at different temperatures.

FIG. 13 shows N-acetylglucosamine hydrolysis with DOW MONOSPHERE 88™ resin in the hydrogen form.

FIG. 14 shows N-acetylglucosamine hydrolysis with DOW MONOSPHERE 88™ resin in the hydrogen form at 90° C.

FIG. 15 shows a comparison of the N-acetylglucosamine hydrolysis shown in FIGS. 14 and 15.

FIG. 16 shows a time-course of N-acetylglucosamine hydrolysis using AMBERLYST 119™ resin according to a method of the present invention.

FIG. 17 a time-course of N-acetylglucosamine hydrolysis using DOW 650C™ resin according to a method of the present invention.

FIG. 18 shows time-course of NAG hydrolysis and acetate formation in a NAG sample applied to an AMBERLYST 119™ resin according to a method of the present invention.

FIG. 19 shows the profile of glucosamine elution from the AMBERLYST 119™ resin according to a method of the present invention.

### DETAILED DESCRIPTION OF THE INVENTION

The present invention is drawn to methods of solid acid hydrolysis of N-acetylglucosamine (NAG) to glucosamine. These methods greatly reduce the amount of excess acid necessary to hydrolyze NAG, thereby reducing or eliminating the need for acid recycle facilities and reducing or eliminating the use of activated carbon to decolorize the hydrolysis solution. The number of crystallization steps required to produce high quality glucosamine are also reduced, which improves the production yields of glucosamine.

The present invention incorporates the use of cationic exchange media in the hydrogen ion form to hydrolyze NAG to glucosamine, releasing acetic acid. The glucosamine free base then binds to the active acid sites on the resin, and can be released by passing an acid over the media to combine with the free base and produce the neutral, stable salt, while regenerating the cationic exchange media for reuse.

The hydrolysis reaction takes place on the cationic media using water to hydrolyze the acetyl bond, releasing acetic acid to the liquid solution, while adsorbing the glucosamine free base on the cationic media where degradation reactions that occur during homogeneous acid hydrolysis are decreased. Moreover, with the free base tightly bonded to a solid substrate, the media can be thoroughly washed to reduce impurities present in the NAG. This mechanism increases yield of the glucosamine product by decreasing degradation and reducing further purification processing steps.

The method includes contacting NAG with a cationic exchange resin in an aqueous medium where the acetyl group is hydrolyzed from the N-acetylglucosamine, and the glucosamine is eluted from the resin to produce the free amine or a stable salt of glucosamine for sale or for further processing. The NAG starting material can be any suitable NAG source that will provide sufficient contact with the cation exchange resin. Although NAG is available commercially from many chemical vendors, such as SIGMA™ (St. Louis, Mo., USA) and nutraceutical ingredient suppliers such as DNPTM (Whittier, Calif., USA), the NAG starting material need not be perfectly pure. A preferred source is NAG formed by fermentation in a fermentation medium as described in U.S. Pat. No. 6,372,457, the entirety of which is incorporated herein by this reference. NAG formed by such fermentation methods may be partially purified to remove cellular material, biomass, cations and anions. It may also be concentrated to reduce the hydrolysis reaction time. The starting concentration of the NAG may be any concentration of NAG that remains soluble under hydrolysis reaction temperatures. Typically, the NAG starting material has a concentration between about 10% by weight and about 70% by weight. Preferably, the NAG starting material has a concentration between about 20% by weight and about 50% by weight. More preferably, the NAG starting material has a concentration between about 20% by weight and about 30% by weight.

The ion exchange resin used should be a strong cation exchange resin. Numerous suitable resins are available commercially including DOW MONOSPHERE 88™ (D-88), DOW M-31™ (M-31), DOW C-575™ (D-575), Rohm & Haas AMBERLYST 16™ (A-16), Rohm & Haas AMBERLYST 39™ (A-39), Rohm & Haas AMBERLYST 119™ (A-119), AND DOW 650C™ (D-650C). The hydrolysis can be conducted in a mixed bed of resin and aqueous NAG, or by recirculating the hydrolysis solution over an ion exchange column until a satisfactory level of conversion has been achieved. If desired, this can be conducted while removing water and acetic acid simultaneously by evaporation to concentrate the solution in contact with the resin, thereby maintaining higher reaction rates. Further, if desired, the step of contacting the NAG with an ion exchange resin can be done in the presence of a gas, including, but not limited to, nitrogen gas.

The molar ratio of the resin functional groups (most often sulphonic acid groups) to the NAG starting material should be about 1:1 or higher. Preferably, the molar ratio of the resin functional groups to the NAG starting material is about 2:1 or higher. More preferably, the molar ratio of the resin functional groups to the NAG starting material is between about 1:1 and about 5:1. More preferably, the molar ratio of the resin functional groups to the NAG starting material is between about 2:1 and about 3:1.

The NAG solution is exposed to the resin for a period of between about 15 minutes and about 36 hours depending upon the hydrolysis rate of the NAG, the desired yield and processing time, as well as cost and processing control considerations such as the amount of resin used and the purity and concentration of the NAG starting material. Generally, the longer the exposure to the cation exchange resin, the greater the hydrolysis of NAG to glucosamine. But at starting NAG concentrations of about 40% and lower, most of the NAG hydrolysis takes place in the first three hours of exposure to the resin and the processing conditions can be controlled such that the majority of the hydrolysis takes place in the first hour of exposure to the resin. Thus, the time of NAG exposure to the cation exchange resin is typically between about 30 minutes and about four hours. Preferably, the time of NAG exposure to the cation exchange resin is between about 30 minutes and about 100 minutes. More preferably, the time of NAG exposure to the cation exchange resin is about 60 minutes.

The hydrolysis may be conducted at a temperature between room temperature and temperatures at which the degradation of NAG and the glucosamine product significantly reduce the yield of the product. Generally, the higher the temperature, the faster the hydrolysis reaction occurs and the more complete is the hydrolysis of NAG for any given period of time. Typically, the hydrolysis of NAG is conducted at a temperature between about 20° C. and about 150° C. Preferably, the hydrolysis is conducted at a temperature between about 50° C. and about 130° C. More preferably, the hydrolysis is conducted at a temperature between about 90° C. and about 120° C. More preferably, the hydrolysis is conducted at a temperature between about 100° C. and about 115° C. More preferably, the hydrolysis is conducted at a temperature between about 107° C. and about 113° C. Most preferably, the hydrolysis is conducted at a temperature of about 110° C.

At elevated temperatures, the hydrolysis components in aqueous solution should be kept under pressure to prevent evaporation and dehydration and the subsequent concentration of the reactions components. At the elevated temperatures used, a reaction pressure of about three to about fifty psig is generally sufficient. Preferably, the hydrolysis reaction is carried out under a pressure of between about 3 psig and about 10 psig. More preferably, the hydrolysis reaction is carried out under a pressure of about 5 psig.

At an elevated temperature, it is possible to vent water and acetic acid vapor from the reaction as the NAG hydrolysis proceeds. This serves to drive the hydrolysis reaction further to completion and reduce the needed number of washing steps, if any, employed before elution, as described below. The released vapor is captured and condensed and neutralized before disposal. The pressure and temperature should be monitored and adjusted to prevent the dehydration of the reaction and drying of the resin. Preferably, the resin is kept submerged in the aqueous reaction solution when the reaction is conducted in a resin bed.

As the hydrolysis reaction proceeds, the pH of the aqueous medium typically drops below about 5. However, the reaction can be successfully conducted at a pH between about 2.0 and about 5.0. Preferably, the hydrolysis reaction is carried out between about pH 2.0 and about pH 4.5.

After the hydrolysis reaction has proceeded under the desired conditions and for a suitable length of time, the glucosamine is eluted off the cation exchange resin by contacting the resin with any cation-containing solution. Preferably, high-activity cations are used to displace the glucosamine, which is ionically attracted to the resin. The glucosamine is stabilized and eluted from the resin by forming its salt (e.g. Glucosamine-HCl, Glucosamine₂-H₂SO₄, Glucosamine₂-H₂SO₄—(KCl)₂, Glucosamine₂-H₂SO₄—(NaCl)₂, Glucosamine₂-NaHSO₄—(HCl)₂, Glucosamine₂-KHSO₄—(HCl)₂), the choice of eluent depends upon the desired product. Suitable salts include sodium chloride, potassium chloride, sodium sulfate, potassium sulfate, sodium bisulfate, potassium bisulfate, hydrochloric acid, sulfuric acid, or combinations of these salts. Preferably, the glucosamine is eluted from the resin by contacting the resin with hydrochloric acid. The hydrochloric acid solution used for elution typically has a concentration between about 0.5N and about 4N. Preferably, the concentration of the hydrochloric acid elution solution is about 2N.

The elution can be conducted with multiple washes of the desired acid or salt solution to progressively remove more glucosamine product from the resin. Generally, the majority of the glucosamine product is removed from the resin with the first wash of acid or salt solution, and that wash is conducted for less than about four hours. However, to elute additional glucosamine from the resin, the resin may be submerged in a fresh solution of acid or salt and soaked over night. These elutions may be conducted at room temperature or an elevated temperature, such as the temperature at which the hydrolysis reaction was conducted. For hydrolysis reactions conducted on a column of resin, the glucosamine product is typically washed with the acid or salt solution several times to assure sufficient recovery of glucosamine. Preferably, the column is washed with an acid or salt solution between about two and about 20 times. More preferably, the column is washed with an acid or salt solution between about three and about 10 times.

The resin may be optionally washed with water before the elution of glucosamine with and acid or salt solution. The water washes generally do not remove any appreciable amount of the glucosamine product from the resin, but are effective in removing nearly all of the remaining unreacted NAG starting material and hydrolyzed acetate from the resin. Although any number of such water washes can be conducted, at least two and preferably, three to five water washes are typically sufficient to remove nearly all of the remaining reaction components with the exception of the glucosamine product bound to the resin.

Non-hydrolyzed N-acetylglucosamine recovered in the elution step may be recycled to fresh resin or the next processing batch of NAG for hydrolysis to permit nearly complete conversion of the NAG starting material to glucosamine.

In accordance with processes of the present invention, high yields of glucosamine from NAG can be achieved. As used herein, such a value refers to the percent hydrolysis of NAG by the cation exchange resin and the percent recovery of glucosamine from the resin. In particular, processes of the present invention can achieve at least about 70% hydrolysis of NAG, more preferably at least about 80%, and more preferably at least about 90%. Processes of the present invention can achieve at least about 70% recovery of glucosamine from a cation exchange resin, more preferably at least about 80%, and more preferably at least about 90%. Processes of the present invention can achieve at least about 50% yields of glucosamine from NAG, more preferably at least about 60%, and more preferably at least about 70%.

After elution from the resin, the solution of glucosamine salt may then be concentrated. If crystallization of the glucosamine is desired, a liquid precipitant, such as an aqueous alcohol, can be used to further reduce solubility. The crystals formed are collected, optionally washed in alcohol and dried.

### EXAMPLES

### Example 1

### Stirred Flask Experiments

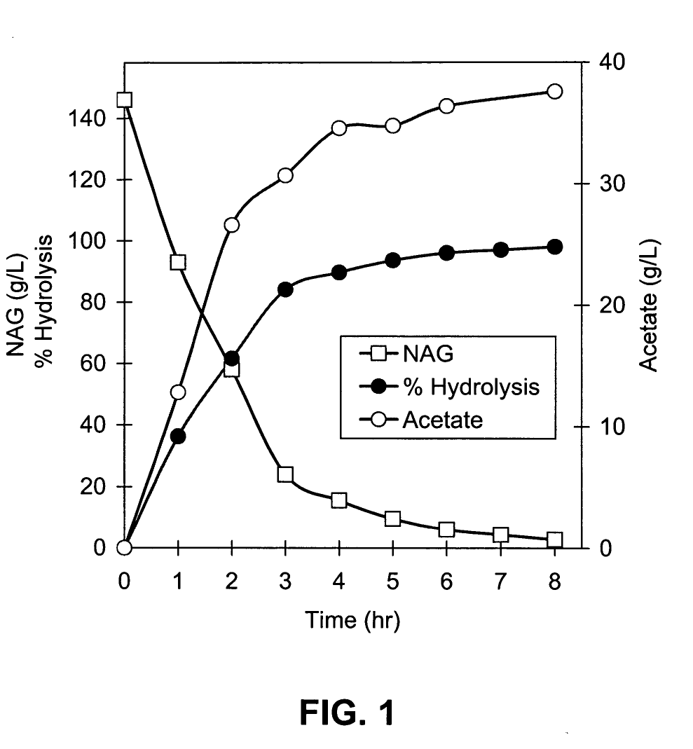

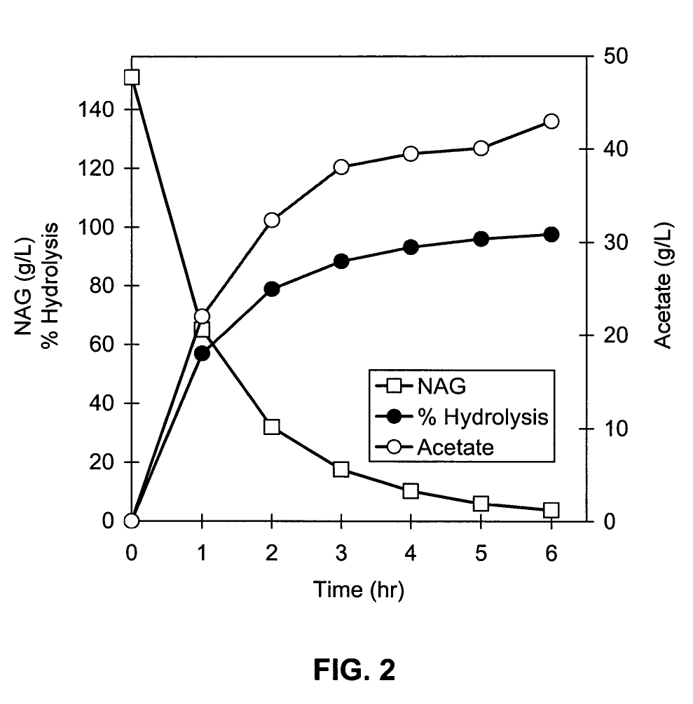

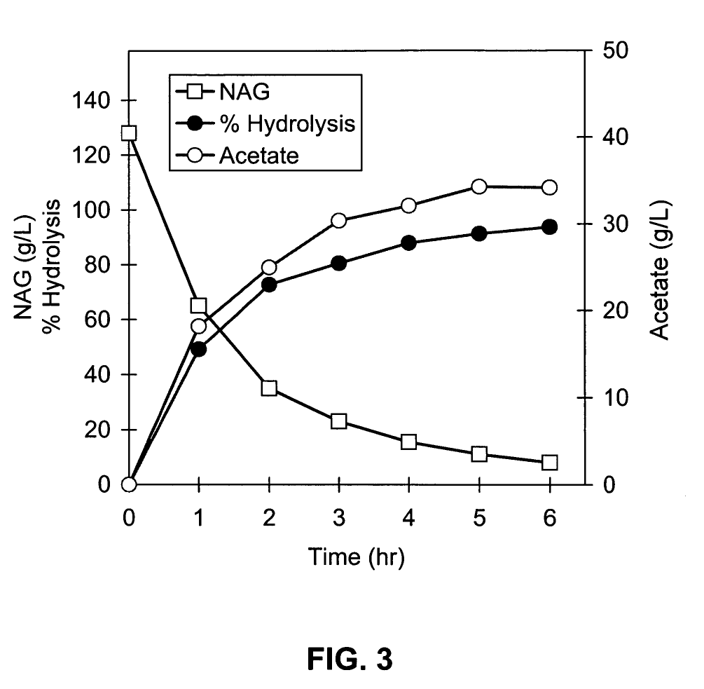

Several experiments were performed using a one-liter round bottom flask fitted with a stirrer and condenser which works well for examining NAG hydrolysis at 1001C. Mixing is excellent, and the condenser prevents significant evaporation. Using this setup, NAG hydrolysis using 125 mL of D 650C 130 mL of A-39 and 125 mL of A-119 resins was examined. In each experiment, 150 mL of 20% NAG was added to the resin volume (drained after measurement). Total volume was measured (about 265 mL in all three cases), and the material was transferred to the one-liter flask. After equipment assembly, a preheated heating mantle was applied to bring flask temperature up to boiling. Flask contents were not at temperature until about 20 minutes after heating was started. Thus, hydrolysis rates during the first hour represent an overall rate observed during this temperature increase. Samples were taken at hourly intervals and analyzed for NAG and acetate. Results for the three resins are shown in FIGS. 1 to 3.

In all three experiments, hydrolysis was much faster during the first two to three hour period. Reaction rates slowed dramatically beyond this as the concentration of NAG decreased and the resin bound more glucosamine. By six hours, reaction rates were approaching zero and hydrolysis was nearly complete. Essentially all glucosamine formed was bound to the resin. Recovered glucosamine HCl only accounted for about 80% of the hydrolyzed NAG, despite extensive washing of reacted resin. Terminating the reaction at two to three hours should give a significantly higher yield. Both AMBERLYST™ resins appear to perform better than the D-650C resin. At two hours, hydrolysis with A-119 (79%) or A-39 (73%) was higher than using the D-650C (62%).

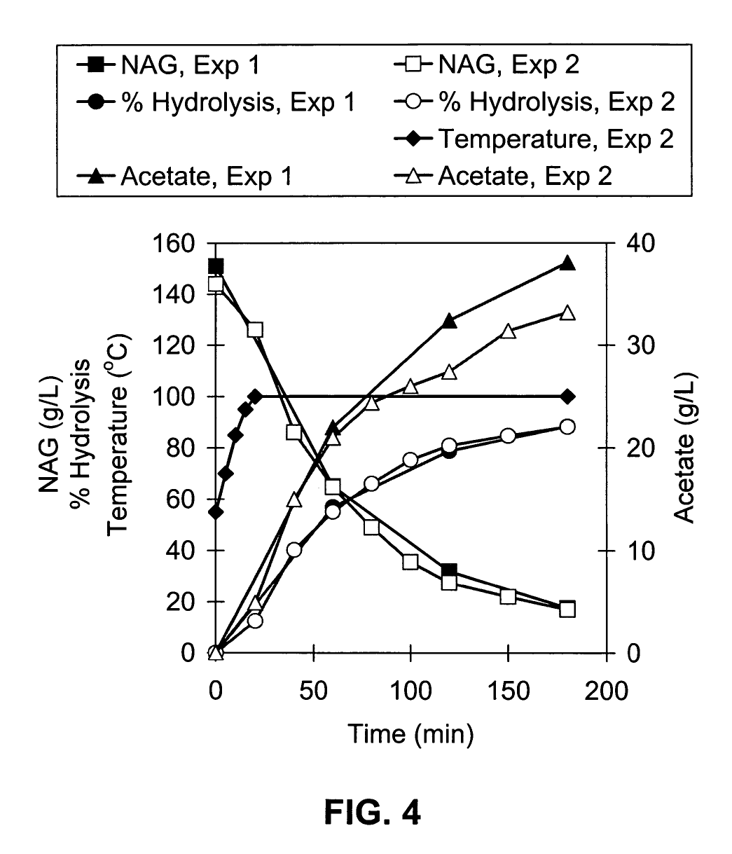

A second experiment using A-119 at 100° C. was performed over a three-hour period, with samples at 20-minute intervals using a one-liter round bottom flask fitted with a stirrer and condenser. A time-course is shown in FIG. 4, which also includes the NAG curve from the first experiment using the A-119 resin. Data from this second experiment compares well to the first experiment. Temperature inside the flask from the second experiment is also shown, and it takes 20 minutes for the reaction to reach 100° C. The time lag in achieving reaction temperature is reflected in the relatively low level of hydrolysis at the 20-minute time-point.

### Example 2

### Unmixed Autoclave Experiments

The effect of temperature on these hydrolysis experiments was evaluated in an autoclave looking at NAG hydrolysis at a temperature of about 125° C. Again, 125 mL resin was mixed with 150 mL 20% NAG in a 500 mL flask. The flasks were held at 125° C. for about 60 minutes. Including heating and cooling cycles, total time at an elevated temperature was about 100 minutes. No mixing was possible in these reactions. The flasks were cooled and assayed for NAG, acetate and glucosamine. In the first experiment, D-650C, D-88, M-31 and A-119 were used. Results are shown in Table 1.

TABLE 1
NAG Hydrolysis at 125° C.

| | % NAG | Yield |
|---|---|---|
| Resin | Hydrolysis | (%) |
| D-650C | 79.3 | 75.8 |
| D-88 | 69.4 | 74.1 |
| M-31 | 58.7 | 68.5 |
| A-119 | 85.3 | 67.7 |

These data mirror those seen at 90° C. Of the three previously tested resins, D-650C is the best although the A-119 resin gave slightly higher hydrolysis. Yields in all four cases were low. This may be due to the high temperature, or less than optimum resin elution. As a control, 20% NAG was autoclaved alone. The solution changed from yellow to orange with autoclaving. About 3% degradation was observed. No acetate was detected.

A second autoclave experiment was performed. The resins used were AMBERLYST™ A-16, A-39, A-119 and D-650C Incubation at 125° C. was for about 50 minutes. Total time in the autoclave was about 90 minutes. Results are shown in Table 2.

TABLE 2
NAG Hydrolysis at 125° C.

| | % NAG | |
|---|---|---|
| Resin | Hydrolysis | Yield |
| A-16 | 71.7 | 85 |
| A-39 | 80.3 | 80.9 |
| A-119 | 81 | 84.8 |
| D-650C | 77 | 77.3 |

Both A-39 and A-119 appear to perform at least as well as D-650C. With only slightly lower levels of hydrolysis, yields were significantly better with this shorter incubation time, as well as a more complete recovery of glucosamine upon acid elution of the resins. A solution of 20% NAG was autoclaved as a control. The solution was visibly darker following autoclaving. About 13.5% loss in NAG was observed. No acetate was detected.

### Example 3

### Thermal Stability of NAG

Thermal stability of NAG at temperatures above 100° C. is an important issue relevant to resin hydrolysis. The stability of NAG was examined in the autoclave using both DNP™ and SIGMA™ NAG as 20% solutions. The DNP™ NAG is described as >98%, SIGMA™ as 99% minimum. This experiment was performed twice. NAG solutions were prepared and autoclaved in sealed serum vials to prevent changes in volume. Total autoclave time was 90 minutes in the first experiment, and 60 minutes in the second experiment. Autoclaved and control samples were analyzed as follows: triplicate identical dilutions of each sample were made; each sample was analyzed three times by HPLC for NAG concentration. This method allowed both measurement of variability in the dilution process, and in HPLC analysis. Results of the two experiments are shown in the Table 3 in which the numbers refer to the percent decrease in the measured concentration of NAG relative to controls kept at room temperature.

TABLE 3
Thermal Degradation of NAG.

| Sample | Experiment 1 | Experiment 2 |
|---|---|---|
| DNP ™ NAG autoclaved | 8.5 | 3.1 |
| SIGMA ™ NAG autoclaved | 18.8 | 15.6 |

Both NAG sources turned orange with autoclaving. Degradation was greater in the first experiment due to the longer heating period.

### Example 4

### Resin Column Experiments

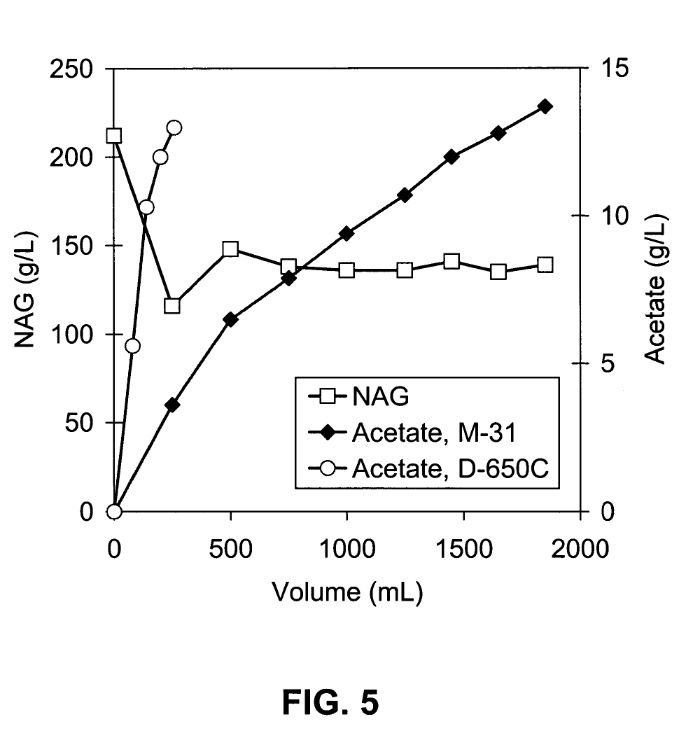

N-acetylglucosamine hydrolysis was also examined using the M-31 resin in a 2.6 cm diameter column containing 125 mL resin at 90° C. by initially pumping two bed volumes per hour of 20% NAG. All eluant was collected in a reservoir held at 90° C. This material was then passed through the column again. In total, the solution was passed through the column eight times, with samples taken after each passage. Results are shown in the FIG. 5. Acetate concentration increased throughout the experiment, indicating continuing hydrolysis was occurring. After the last pumping cycle, the column was washed with water, then 2 N HCl. These samples were analyzed for acetate, N-acetylglucosamine, and glucosamine. Results are shown in Table 4 in which the numbers indicate grams of the chemical and “ND” refers to a value that was not determined.

TABLE 4
NAG Hydrolysis Reaction Components.

| | Volume | NAG | Glucosamine · HCl | Acetate |
|---|---|---|---|---|
| Sample | (ml) | (g) | (g) | (g) |
| Final pump sample | 180 | 25 | ND | 2.5 |
| Water wash | 220 | 12.7 | ND | 1.3 |
| HCl wash | 295 | 0.0 | 13.9 | 0.0 |

About 260 mL 20% NAG (52 g) was loaded onto the column. Based on disappearance of NAG, about 14 g was hydrolyzed in eight hours (27%). The amount of acetate measured is 100% of the expected amount based on NAG measurements. The amount of glucosamine•HCl formed is essentially 100% of expected. Thus, there appears to be no degradation of NAG in the column at this temperature. Hydrolysis on M-31 resin appears to be slow. Because the design of this experiment was different from the D-650C run, we really cannot directly compare the experiments. Protocol for the first 250-mL region of both experiments is the same, however.

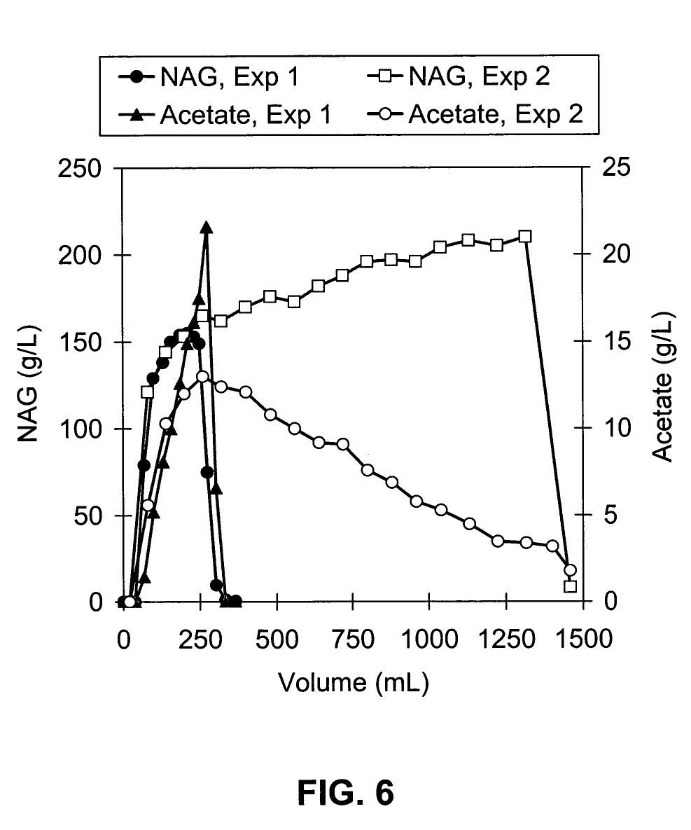

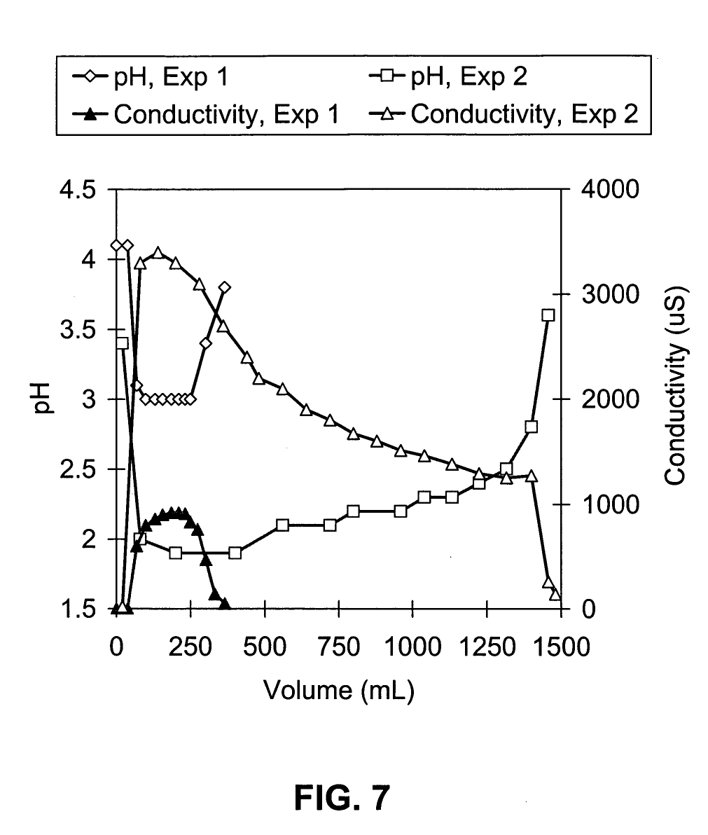

N-acetylglucosamine hydrolysis using the D-650C resin in column format was evaluated on a column having a dimension of 2.6 cm in diameter with a bed depth of about 23 cm. Bed volume was 125 mL. Column temperature was maintained about 90° C. using a water jacket. Heated solutions were pumped through the column at about 250 mL per hour. The planned format of the experiment was to pump 2500 mL NAG (20%), 250 mL water, and 250 mL 2N HCl. Because of leakage and the length of the experiment, only two liters 20% NAG were pumped, with an estimated 1.4 liters actually passing through the column. Results are shown in FIGS. 6 and 7. Also plotted are data from the first column reaction experiment. Column pH dropped to about 2.0 as N-acetylglucosamine began to be hydrolyzed. For unclear reasons, this drop was greater than that seen in Experiment 1. Initial conductivity increase was also much greater than in the previous experiment. Acetate levels decreased, but were still about 3 g/L when the N-acetylglucosamine feed was discontinued. Efflux NAG concentration at the end of the run was about 210 g/L. Input NAG concentration, as measured, was 214 g/L. The pH gradually rose, but was still lower than the 4.4 pH of the input stream. Conductivity gradually decreased, but was still above the input value of 830 μS. FIGS. 6 and 7 indicate that N-acetylglucosamine is still being hydrolyzed at the end of the run. Column reactivity is seen to deteriorate, but the column does not become completely deactivated. Washing with 2 N HCl released 37 g glucosamine•HCl (0.17 moles). This converts to 1.37 moles glucosamine per liter of resin. Thus the amount of glucosamine that can bind during the hydrolysis reaction appears to be at least 1.5 equivalents/liter resin.

N-acetylglucosamine hydrolysis using the D-650C resin in column format was performed on a column having dimensions of 2.6 cm in diameter with a bed depth of about 23 cm. Bed volume was 125 mL. Column temperature was maintained at about 90° C. using a water jacket. Heated solutions were pumped through the column at about 250 mL/hour. The planned format of the experiment was to pump 250 mL NAG (20%), 250 mL water, 250 mL 2N HCl. Additional HCl was pumped through the column to ensure complete elution of glucosamine. After the experiment was completed, the resin was removed and treated with 2N HCl overnight as well. Column eluant was collected in fractions during the N-acetylglucosamine and water feeds. The column was cooled and the eluant from the HCl feed was collected in a container. After pumping, remaining liquid in the column was withdrawn by syringe and added to the HCl wash. Because of leakage, the total volume of the fractions collected was less than 500 mL.

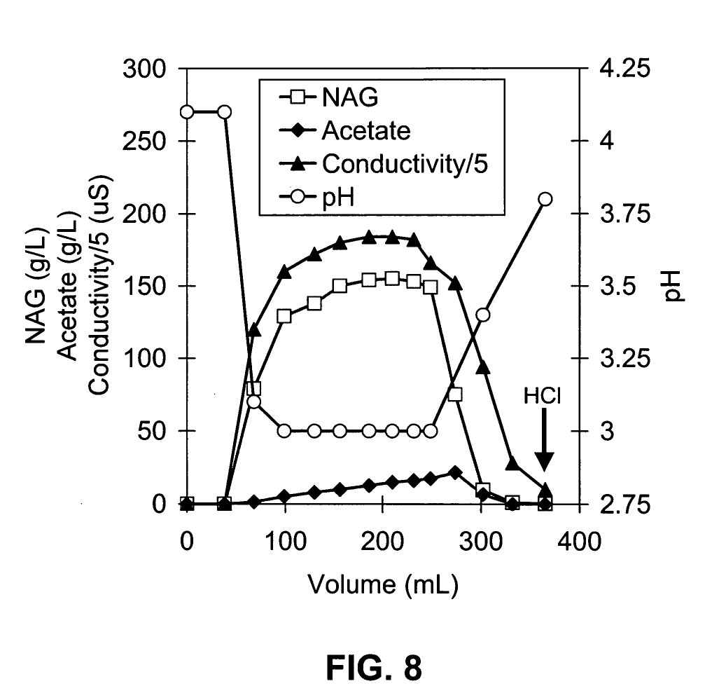

FIG. 8 shows concentrations of N-acetylglucosamine and acetate, as well as pH and conductivity during the N-acetylglucosamine feed and water wash. Significant N-acetylglucosamine passed through unhydrolyzed. Conductivity increased during the N-acetylglucosamine feed, and returned essentially to zero with the water wash. The pH decreased with the N-acetylglucosamine feed, and returned to a higher value with the water wash. Assuming there was no glucosamine in the fractions collected during the N-acetylglucosamine and water wash, in the first 415 mL HCl wash, 25 g/L glucosamine•HCl was found. No glucosamine was detected in the overnight wash. The recovered yield of glucosamine•HCl was 10.4 g, or 0.048 moles, and is 19.2% of the manufacturer's rating of 2 eq/l.

The material balance of the column hydrolysis reaction was evaluated for N-acetylglucosamine hydrolysis using the D-650Cresin. Reaction conditions were similar to those described previously (90° C. in jacketed column with nitrogen sparging, 220 mL resin volume, 30 g NAG added in about 300 mL water). The batch reaction was carried out for about 6.5 hours. No time-point samples were taken. After cooling the column, the hydrolysate was drained, and the resin washed with 250 mL water. These washes were combined (water wash 1). The resin was washed for three hours with 2N HCl (HCl wash 1), and further washed overnight with 2N HCl (HCl wash 2). These three samples were analyzed for N-acetylglucosamine, glucosamine, and acetate. Results are shown in Table 5 in which the numbers indicate grams.

TABLE 5
Mass Balance of Column Hydrolysis Reaction.

| | Volume | NAG | Glucosamine · HCl | Acetate |
|---|---|---|---|---|
| Sample | (ml) | (g) | (g) | (g) |
| Water wash 1 | 550 | 6.6 | 0.0 | 4.6 |
| HCl wash 1 | 465 | 0.0 | 18.6 | 0.0 |
| HCl wash 2 | 265 | 0.0 | 3.2 | 0.0 |

From the table, 23.4 g N-acetylglucosamine was hydrolyzed (78%). This should yield 22.8 g glucosamine•HCl. A total of 21.8 g (95.6%) could be accounted for. Observed acetate was low at only 72.5% (4.6/6.3) of the expected value based on remaining N-acetylglucosamine. Material balance indicates that N-acetylglucosamine hydrolysis using cation resins results in less degradation and a much cleaner glucosamine product than that seen using HCl.

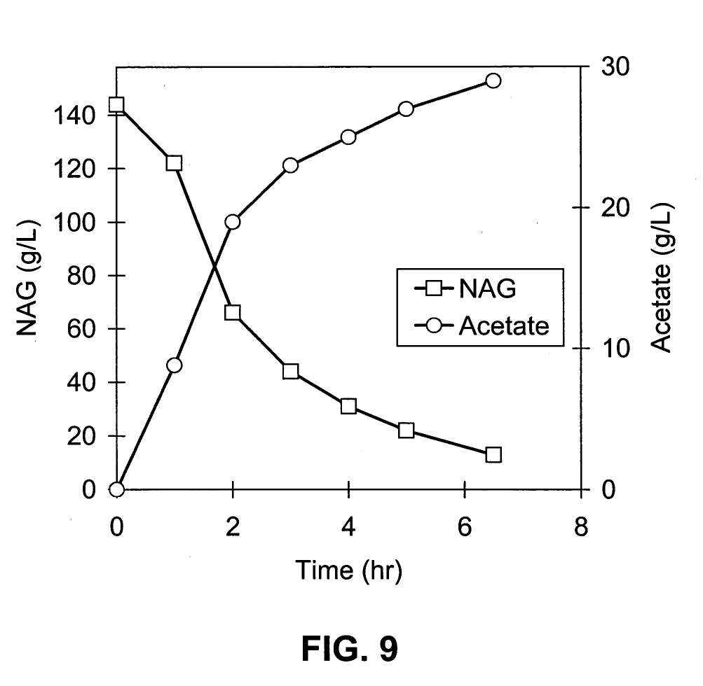

The time-course of N-acetylglucosamine hydrolysis on the D-650C resin was examined using reaction conditions similar to the previously described experiments (88° C. in jacketed column with nitrogen sparging, 230 mL resin volume, 150 mL of 20% NAG). FIG. 9 shows the time-course for N-acetylglucosamine disappearance and acetate formation. Hydrolysis was initially fairly rapid, with about 50% hydrolysis after two hours. Beyond this point, hydrolysis began to slow, with about 90% hydrolysis at 6.5 hours. The D-650C resin is described as having 2 eq/l. This may be high, but even using 1.5 eq/l, the resin was significantly in molar excess of the amount of N-acetylglucosamine (30 g) present. Resin efficiency decreases as more glucosamine is formed.

The mass balance of this reaction was examined using similar reaction conditions (N-acetylglucosamine (30 g) in 200 mL water was added to 225 mL of the D-650C resin). Notice-course samples were taken. After 6.5 hours, the reaction was allowed to cool to room temperature. The hydrolysate was drained, and the resin was washed with water. These two fractions were combined before assay (Water wash 1). The drained and washed resin was treated for 30 minutes with 250 mL of 2 N HCl (HCl wash 1). The resin was agitated by nitrogen sparge. This acid wash treatment was repeated twice (HCl washes 2,3), followed by an overnight incubation in 2 N HCl (HCl wash 4). These five samples were assayed by HPLC for N-acetylglucosamine, acetate and glucosamine. Results are shown in Table 6 in which the numbers indicate grams of the component, and “ND” indicates values that were not measured.

TABLE 6
Mass Balance of N-acetylglucosamine Hydrolysis Reaction.

| | Volume | NAG | Glucosamine (free) | Acetate |
|---|---|---|---|---|
| Sample | (ml) | (g) | (g) | (g) |
| Water wash 1 | 440 | 4.1 | 0.0 | 5.0 |
| HCl wash 1 | 250 | 0.1 | 11.9 | 0.2 |
| HCl wash 2 | 250 | ND | 4.5 | ND |
| HCl wash 3 | 235 | ND | 1.6 | ND |
| HCl wash 4 | 265 | ND | 0.8 | ND |

From the table, 25.8 g N-acetylglucosamine was hydrolyzed during the experiment (86% of initial amount added). Total hydrolysis of this amount of N-acetylglucosamine would give a theoretical yield of 21 g free glucosamine and 7 g acetic acid. The actual amounts detected were 18.8 g glucosamine (89.5%) and 5.2 g acetate (74.3%). Some acetic acid was lost as vapor because of an inefficient condenser. Assuming these numbers are accurate, there appears to be some degradation of glucosamine during the reaction. Presumably, degradation accelerated during the later part of the experiment. The water hydrolysate is light yellow in appearance. The HCl washes are all colorless, suggesting the type of degradation occurring is different than that seen using HCl. From the presence of significant glucosamine, even in the fourth HCl wash, it is clear that to fully elute glucosamine, the resin must be in contact with acid for a significant length of time, or the acid should be passed over the resin as for a fixed-bed elution.

### Example 5

### FPLC Column Hydrolysis

Experiments using a 5-cm FPLC Pharmacia column were conducted in which mixing was provided by bubbling nitrogen in through the bottom port. Temperature was maintained at about 88° C. using a circulating water bath pumping through the column jacket. Reactions contained about 230 mL resin mixed with 150 g of 20% N-acetylglucosamine. Since solutions were added at room temperature, there was a 15 to 20 minute lag until the reaction mixture reached the final temperature. Mixing was vigorous in the top portion of the chamber, but there was some settled resin on the bottom, which was continuously lifted into the upper region of the reactor. Lack of an efficient condenser system undoubtedly resulted in some volume loss due to evaporation over the seven-hour reaction period and product concentrations measured are, therefore, probably 10 to 15% too high in the later time points.

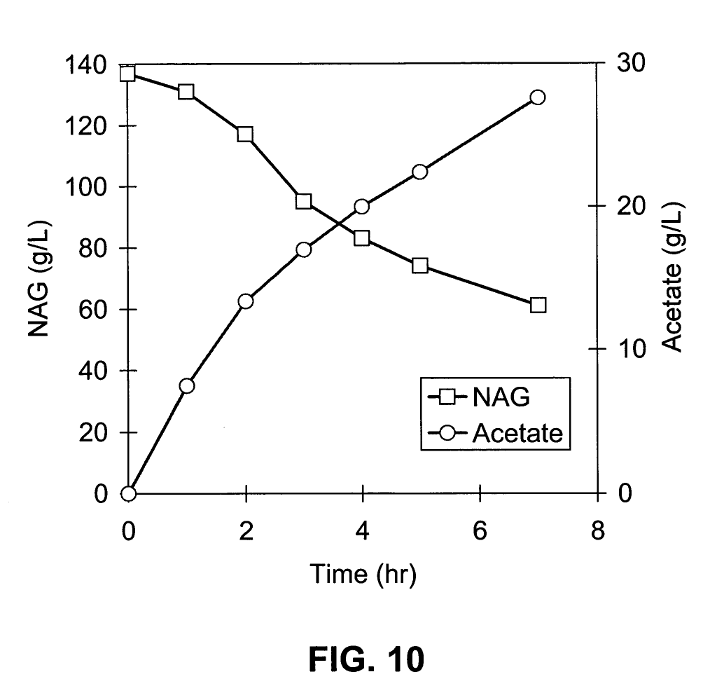

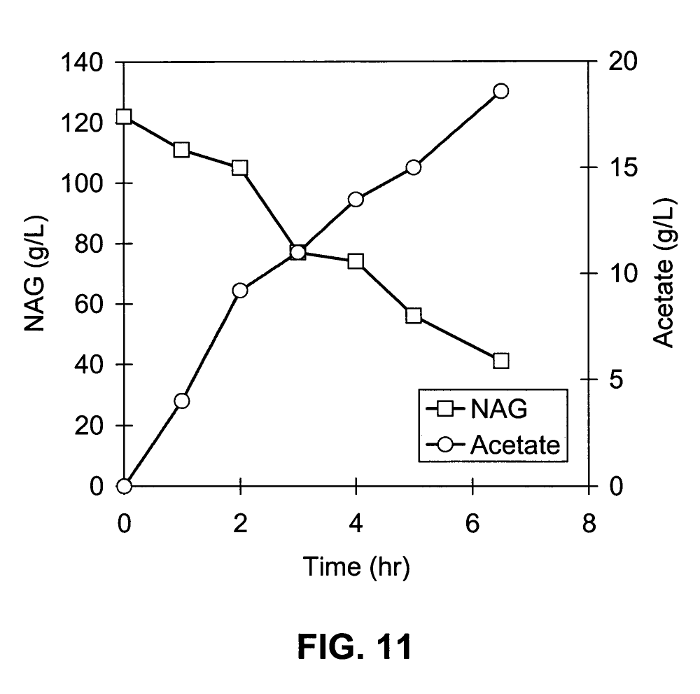

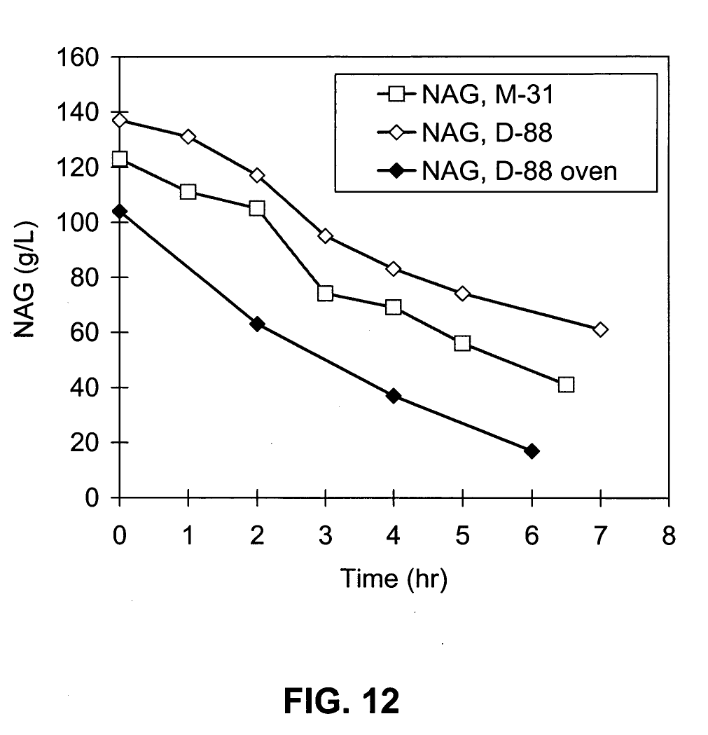

FIG. 10 shows results using the D-88 resin. Disappearance of N-acetylglucosamine appears to lag somewhat at the start, presumably due to the initial heating of the reaction mixture. As the reaction progresses, the number and concentration of available resin sites decline, reducing the observed hydrolysis reaction rate. At seven hours, about 55% of the N-acetylglucosamine is hydrolyzed based on the NAG concentration measured by HPLC. This value probably underestimates the actual level of hydrolysis. Note that the measured acetate concentration at seven hours is too high, presumably due to evaporation. FIG. 11 shows hydrolysis using the M-31 resin. At 6.5 hours, measured N-acetylglucosamine level had dropped about 66%. Roughly 82 g/L N-acetylglucosamine was hydrolyzed. This should yield about 15.8 g/L acetate. An actual value of 18.6 g/L was measured. Evaporation has skewed values to show a higher concentration than theoretical. FIG. 12 shows a comparison of hydrolysis of N-acetylglucosamine using the D-88 and M31 resins, as well as a D-88 experiment conducted in a hybridization oven. From these results, use of the column reactor with gas mixing does not appear to accelerate the reaction. Resin and supernatant from the M-31 reaction was recovered and examined for glucosamine. About 0.8 g/L was present in the supernatant. Resin samples were either washed several times with deionized water or used directly after removal of supernatant fluid. Results were essentially the same in both cases. Glucosamine was not removed by washing with water. Treatment with either 1 M NaCl or 1 N HCl released approximately equal amounts of glucosamine from the resin. Roughly 5 g resin washed with 2.5 mL salt or acid gave a solution of about 25 to 30 g/L glucosamine.

### Example 6

### Resin in the Hydrogen Form

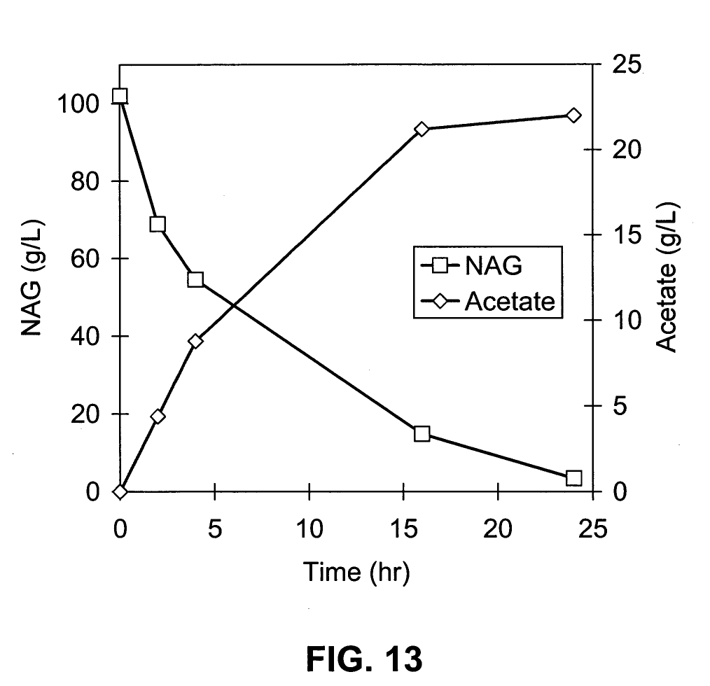

N-acetylglucosamine was incubated at elevated temperature with Dow D-88 resin in the hydrogen form. The resin was prepared by extensive washing first in 2 N HCl, followed by thorough washing with deionized water. The pH of the resin slurry was about 2.7. Approximately 130 grams of wet resin was added to a screw-cap glass tube. Following this, a solution of 20% N-acetylglucosamine was added. The glass tube was incubated with agitation and samples were removed over a 24-hour period. N-acetylglucosamine and acetate were measured by HPLC. In these experiments, the tube and contents were initially at room temperature and the reaction mixture reached the incubation temperature within about 30 minutes. Initial N-acetylglucosamine concentration was about 102 g/L. The tube was incubated at 83° C. Results are shown in FIG. 13. Roughly 50% of the N-acetylglucosamine was hydrolyzed by four hours. After 24 hours, N-acetylglucosamine was detected at about 3.5 g/L. The amount of acetate at 24 hours was about 22 g/L.

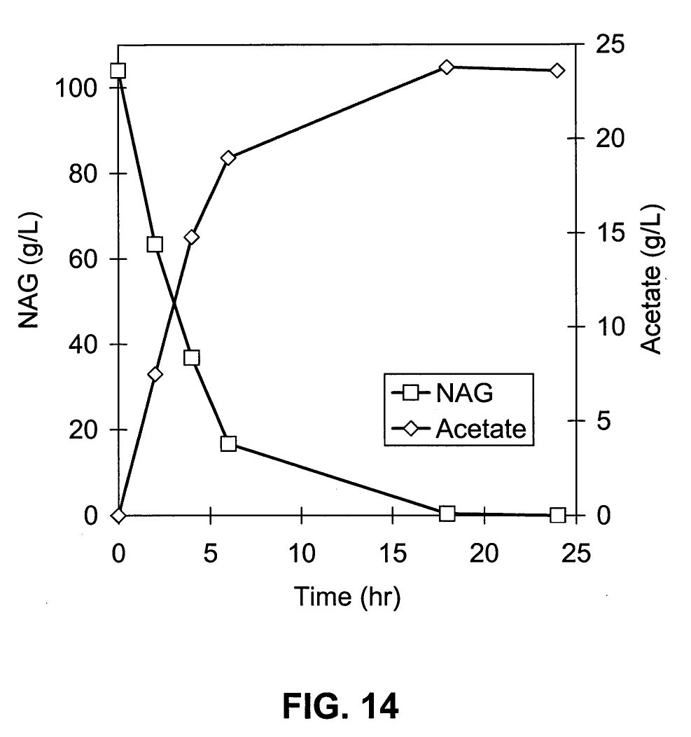

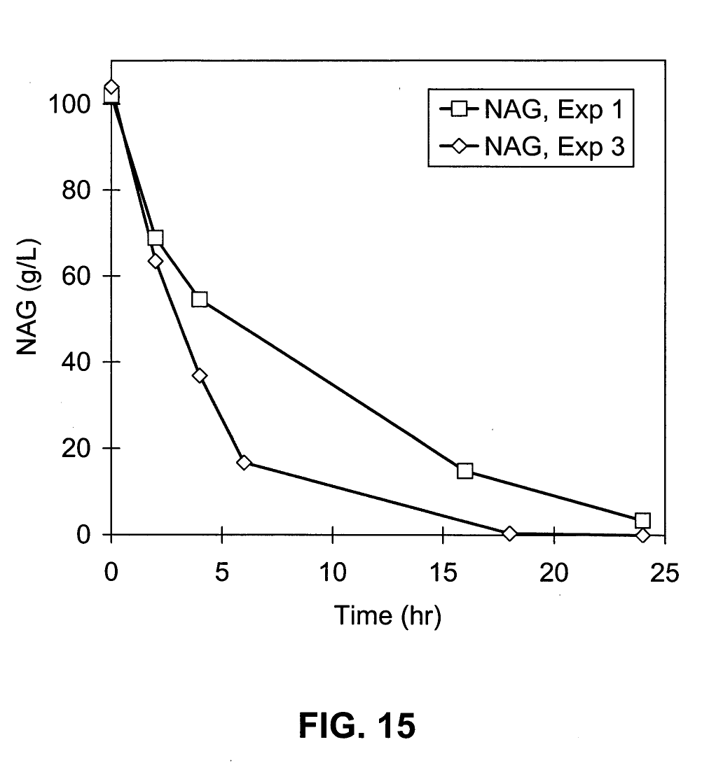

In another experiment, initial N-acetylglucosamine concentration was about 104 g/L. The tube was incubated at 90° C. Results are shown in FIG. 14. Hydrolysis proceeded to completion in this experiment. FIG. 15 compares disappearance of N-acetylglucosamine in these two experiments. Hydrolysis was clearly faster in the second experiment. This may be due to temperature, or may be due to differences in resin levels.

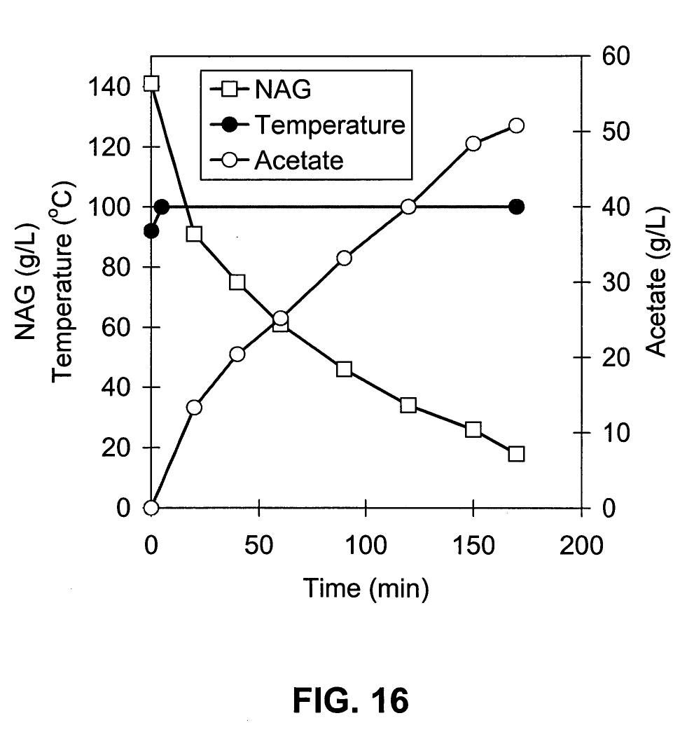

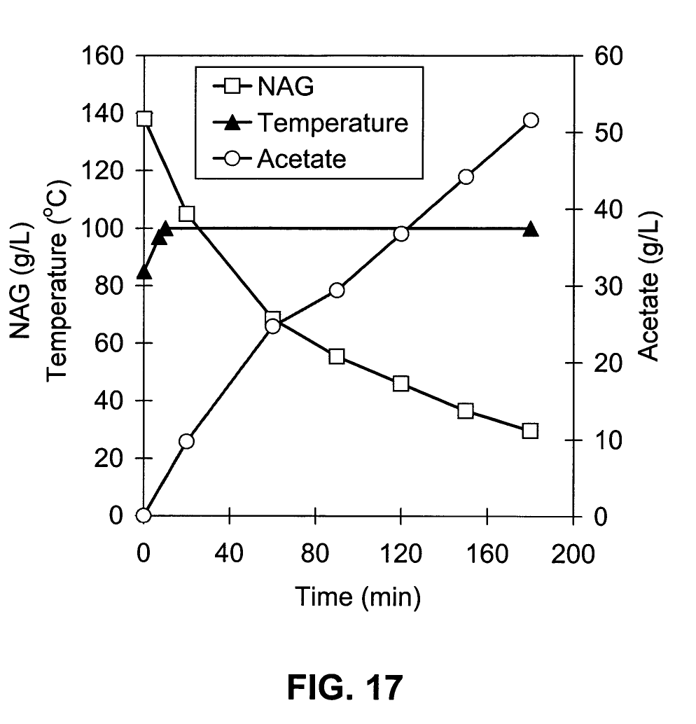

Two additional experiments were done using a one-liter, round-bottom flask fitted with a stirrer and heating mantle. In these experiments, the condenser was replaced by tubing leading to a sidearm flask in an ice bucket. A significant decrease in total volume was apparent in both experiments. The A-119 experiment was terminated at 170 minutes with very little liquid remaining. Trapped condensate was not measured. In another experiment using the D-650C resin, about 120 mL liquid was found in the vapor trap. Due to volume changes, the amount of conversion of NAG to glucosamine•HCl must also reflect changes in solution volume in the reactor. In both experiments, 125 mL of wet resin was used. Preheated NAG solution (150 mL of 20% NAG in water) at 90° C. was added to the heated resin. In the A-119 experiment, 100° C. was reached in five minutes. In the D-650C resin experiment, 100° C. was reached in 10 minutes. FIG. 16 shows the time-course using A-119. Remaining NAG in the reaction was measured as 2.1 g (7%). Thus actual hydrolysis at 170 minutes was 93%. Recovered glucosamine-HCl after resin elution with HCl solution was 24.3 g. This is 89.7% of the 27.1 g formed by hydrolysis. In the experiment using the D-650C resin in the same setup, total remaining NAG was 2.74 g. Thus 90.9% of the 30 g NAG starting material was hydrolyzed. These results are shown graphically in FIG. 17.

Another experiment was done using a one-liter, round bottom flask fitted with a stirrer and heating mantle. The condenser was replaced by a short length of tubing leading to a condenser. In this experiment, 125 mL A-119 resin was used for hydrolysis of 150 mL 30% NAG at 100° C. Samples (2 mL) were taken at 20-minute intervals. After two hours, the resin was transferred to a column and washed with 3×125 mL water. After this, the column was washed with 300 mL 2 N HCl. As in previous experiments, the experiment was designed to minimize the lag time between initiation of the reaction and when the reaction mixture reaches 100° C. To achieve this, 125 mL of resin was preheated under water with stirring in the reaction flask. The NAG solution was heated to about 90° C. in a water bath. Water was removed by pipette and 150 mL 30% NAG was added to a final volume of 258 mL. Addition of the NAG solution to the flask defines the start of the experiment. In this experiment, 100° C. was achieved at about eight minutes. Note that there is significant water left in the resin, reflected in the 23.7% initial NAG concentration in the reaction mixture. After the 120-minute reaction period, the remaining resin slurry was measured and found to be 155 mL. A small amount of liquid was lost here during the transfer (approximately 5%). The condensate recovered was 92 mL total. This represents about 36% of the initial reaction volume (resin+liquid).

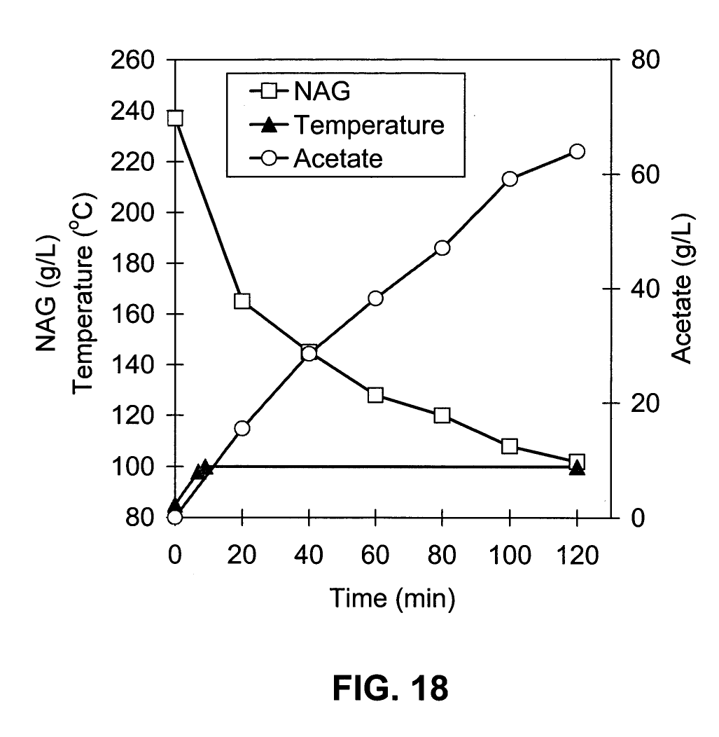

FIG. 18 shows the time-course of NAG hydrolysis and acetate formation. Table 7 summarizes NAG, acetate and glucosamine•HCl values (in grams) obtained for the water and HCl washes of the resin (“ND” indicates values that were not determined). Total NAG recovered was 8.6 g (6.9 g in water washes+1.7 g in time-point samples). Thus 36.4 g (45−8.6) NAG was hydrolyzed (80.9%). This corresponds to 35.5 g glucosamine•HCl. The total amount of glucosamine•HCl recovered from the resin was 30.9 g. This is 87.3% of the amount formed. Because of an accidental spill during a transfer, actual recovery was probably better. Allowing the reaction volume to decrease appears to facilitate higher levels of hydrolysis. In two hours, 0.165 moles NAG was hydrolyzed using 125 mL (0.225 eq) resin. This is more hydrolysis than in three hours using lower NAG concentration without drawing off water.

TABLE 7
Mass Balance of NAG, Acetate and Glucosamine · HCl.

| | Volume | NAG | Acetate | Glucosamine · HCl |
|---|---|---|---|---|
| Sample | (ml) | (g) | (g) | (g) |
| Water wash 1 | 138 | 6.5 | 5.3 | ND |
| Water wash 2 | 125 | 0.4 | 0.6 | ND |
| Water wash 3 | 125 | 0 | 0 | ND |
| HCl 1 | 30 | 0 | 0 | 0 |
| HCl 2 | 29 | ND | ND | 1.16 |
| HCl 3 | 29 | ND | ND | 7.74 |
| HCl 4 | 30 | ND | ND | 8.88 |
| HCl 5 | 31 | ND | ND | 7.38 |
| HCl 6 | 30 | ND | ND | 3.66 |
| HCl 7 | 31 | ND | ND | 1.51 |
| HCl 8 | 31 | ND | ND | 0.45 |
| HCl 9 | 29 | ND | ND | 0.13 |
| HCl 10 | 26 | ND | ND | 0.0 |
| HCl 11 | 26 | ND | ND | 0.0 |
| Condensate | 92 | 0.0 | 6.9 | ND |

### Example 7

### Hydrolysis Under Pressure

To evaluate reactions at pressures above 1 atmosphere, an experiment was done using a stirred autoclave reactor. The reactor was modified by extending the drive shaft and using a larger impeller. A backpressure regulator was also installed. A hydrolysis experiment was performed using 125 mL D-650C resin mixed with 90 mL 30% NAG. Total volume was 190 mL. Agitation was at 500 rpm. The reaction was run at about 110° C. for 70 minutes. There was an initial temperature spike to about 127° C. This was quickly adjusted by pressure release to about 110° C. Temperature was maintained in the range of 107° C. to 115° C. for the remainder of the experiment. The reaction was quickly brought to room temperature at 70 minutes using an ice bath. Backpressure was kept at about 6 psig. Time-point samples were only taken at 0 and 70 minutes. About 58 mL condensate was collected. This was too high a removal rate, as the liquid level fell significantly below that of the resin. Resin was also coating the cooling coils (now removed and ports sealed) and headspace surfaces, suggesting a lower agitation rate should be evaluated. The resin and reaction liquid was transferred to a column. This requires washing reactor surfaces to collect resin, etc. The column was washed with water, then 2 N HCl. The results are shown in Table 8 in which the values are in grams and “ND” indicates values not determined. Glucosamine was determined by calorimetric assay.

At the start of the reaction, NAG concentration was about 21.7%. At the end, it was 2.7%. About 1.9 g NAG was recovered, including material eluted from the column, a small amount present in the vapor condensate, and about 0.37 g present in the 0 and 70 minute samples (1.16+0.07+0.28+0.37=1.88). Thus 25.1 g NAG (93%) (27−1.9) was hydrolyzed. This corresponds to 24.5 g glucosamine•HCl. About 85.3% of this was recovered from the resin. The NAG present in the condensate probably reflects some liquid released when pressure was quickly adjusted down due to the early temperature spike. The HCl wash did have some yellow color. Resin appearance after the reaction was unchanged. Over 90% hydrolysis was observed in approximately one hour.

TABLE 8
Analysis of Reaction Samples Using 650C Resin

| | Volume | NAG | Acetate | Glucosamine · HCl |
|---|---|---|---|---|
| Sample | (ml) | (g) | (g) | (g) |
| Water wash 1 | 106 | 1.16 | 3.77 | ND |
| Water wash 2 | 123 | 0.07 | 0.41 | ND |
| HCl wash | 340 | ND | ND | 20.9 |
| Condensate | 58 | 0.28 | 1.75 | ND |

Another experiment was performed using the stirred autoclave reactor using 125 mL A-119 resin mixed with 100 mL 30% NAG. Total volume was 198 mL. Agitation was at 250 rpm. The reaction was run at 110° C.±3° C. for 60 minutes. Heating was manually controlled. About 53 mL condensate was collected (32.6 g/L acetate). This was roughly 25% of the initial reaction volume. Resin remained submerged at the end of the experiment. Initial NAG concentration was 215 g/L. Final NAG concentration was 22 g/L. Final acetate concentration was 69 g/l. The reaction mixture was loaded onto a column and washed with water (250 mL), 2N HCl (250 mL), and water (150 mL). Samples were analyzed for NAG, acetate and glucosamine. Results are shown in Table 9 in which the values are given in grams and “ND” indicates values not determined. Glucosamine was determined by calorimetric assay. About 1.8 g NAG was not hydrolyzed (1.55+0.22 [0′ time-point sample]), leaving 28.2 g (94%) converted to 27.5 g glucosamine•HCl. About 25.3 g was recovered (92%) from the resin.

TABLE 9
Analysis of Reaction Samples Using A-l 19 Resin.

| | Volume | NAG | Acetate | Glucosamine · HCl |
|---|---|---|---|---|
| Sample | (ml) | (g) | (g) | (g) |
| Water Wash | 222 | 1.55 | 5.4 | ND |
| HCl Wash | 500 | 0.0 | 0.32 | 25.3 |
| Condensate | 53 | 0.0 | 1.7 | ND |

### Example 8

### Elution of Glucosamine from Resin

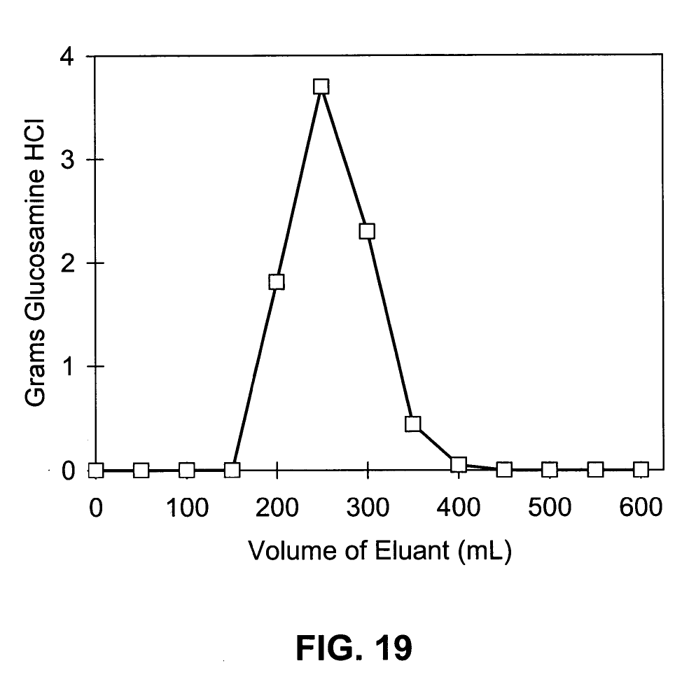

Elution of glucosamine from A-119 resin was examined by applying ten grams glucosamine•HCl in 100 mL water to 125 mL resin. The resin was sequentially washed with 125 mL water, 175 mL 2N HCl, and 300 mL water. Fifty-mL fractions were collected. Glucosamine content was analyzed using the calorimetric assay. The experiment was run at 50° C. Flow rates used were slow, in the range of 1 to 1.5 column volumes/hour. Essentially, all the glucosamine bound to the column. Release with HCl is shown in FIG. 19.

Using 175 mL 2N HCl (1.4 column volumes), about 8.3 g was recovered. The results indicate that additional HCl, probably in the range of two column volumes, is needed for complete elution of bound glucosamine.

### Example 9

### Hydrolysis and Elution of N-acetylglucosamine

Use of a strong cation exchange resin allows hydrolysis of NAG to glucosamine. Preferred reaction conditions include a temperature over 100° C., preferably about 110° C., with a reactor pressure of about 5 psig. The reaction mixture consisting of resin plus an aqueous solution of NAG is agitated at about 200 rpm with a volume of about 200 mL in a small-scale autoclave reactor system. The molar ratio of resin functional groups (sulphonic acid) to NAG is about 1:1, with a preferable ratio of 2:1 or higher. During the reaction, steam and acetic acid may be drawn off from the reactor, thereby reducing reaction volume, increasing the concentration of the remaining non-volatile NAG and helping to drive the hydrolysis reaction further to completion. Following reaction, unreacted NAG may be separated from the resin by washing the resin with water and adding this to the residual reaction fluid. The glucosamine remains firmly bound to the resin, while the NAG is freely eluted. The recovered unreacted NAG may then be mixed with fresh NAG for use in a subsequent NAG reaction cycle, permitting virtually complete conversion of the NAG starting material to glucosamine. The glucosamine bound to the resin during hydrolysis may be eluted using strong mineral acids such as HCl. This allows for simple recovery and purification of the glucosamine as the hydrochloride salt. A number of strong acid cation resins are preferred for the reaction, such as D-650C, A-119, D-575, and similar resins. Use of the food-grade resin D-575, using the reaction conditions described above, allows over 80% hydrolysis of NAG, followed by over 90% recovery of the glucosamine formed. Three such experiments are summarized in Table 10. Three experiments have been performed using 125 mL D-575 resin (2.1 molar equivalents/liter). All three reactions were run at 110° C.±2° C. for 60 to 65 minutes using a molar ratio of resin:NAG of roughly 2:1. Experiments 1 and 2 used commercial NAG. Experiment 3 used NAG from deionized fermentation broth wherein NAG represented 94% of total solids. Percent glucosamine recovered was based on amount of NAG hydrolyzed.

TABLE 10
Heterogeneous N-acetylglucosamine Hydrolysis.

| | Reaction | Reaction | Condensate | NAG | NAG | Glucosamine | Glucosamine |
|---|---|---|---|---|---|---|---|
| | Time | Volume | Volume | Added | Hydrolysis | Formed | Recovered |
| Expt. | (min) | (ml) | (ml) | (g) | (g) | (g) | (%) |
| 1 | 60 | 218 | 63 (29%) | 30 | 25 | 24.5 | 95.2 |
| | | | | | (84%) | | |
| 2 | 65 | 200 | 45 (23%) | 30 | 26.4 | 25.7 | 92.6 |
| | | | | | (89%) | | |
| 3 | 65 | 200 | 57 (29%) | 29.3 | 24.9 | 24.3 | 92.5 |
| | | | | | (85%) | | |

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1. A method of deacetylating N-acetylglucosamine comprising contacting N-acetylglucosamine with a cation exchange resin wherein an acetyl group is hydrolyzed to produce glucosamine.

2. The method of claim 1, wherein the glucosamine becomes bound to the cation exchange resin.

3. The method of claim 1, wherein the cation exchange resin is in the hydrogen ion form.

4. The method of claim 1, wherein the step of contacting is performed at a pH range of about 2 to about 5.

5. The method of claim 1, wherein the step of contacting is performed at a temperature of between about 20° C. and about 150° C.

6. The method of claim 1, wherein the step of contacting is performed at a temperature of between about 100° C. and about 115° C.

7. The method of claim 1, wherein the step of contacting is performed at a temperature of about 110° C.

8. The method of claim 1, wherein the step of contacting is performed under a pressure of between about 3 psig and about 50 psig.

9. The method of claim 1, wherein the step of contacting is performed under a pressure of about 5 psig.

10. The method of claim 1, wherein the molar ratio of resin functional groups to N-acetylglucosamine is between about 1:1 to about 5:1.

11. The method of claim 1, wherein the molar ratio of resin functional groups to N-acetlyglucosamine is between about 2:1 and about 3:1.

12. The method of claim 1, wherein the contacting step comprises drawing off water vapor and acetic acid from a reactor containing the cation exchange resin.

13. The method of claim 1, further comprising eluting glucosamine from the cation exchange resin.

14. The method of claim 13, wherein the step of eluting comprises washing the cation exchange resin with at least one water wash.

15. The method of claim 13, wherein the step of eluting comprises washing the cation exchange resin with a wash solution containing a salt selected from the group consisting of sodium chloride, potassium chloride, sodium sulfate, potassium sulfate, sodium bisulfate, potassium bisulfate and a combination thereof.

16. The method of claim 15, wherein the wash solution comprises at least one of HCl and NaCl.

17. The method of claim 13, wherein the step of eluting comprises washing the cation exchange resin with a wash solution comprising an acid.

18. The method of claim 17, wherein the wash solution comprises at least one of hydrochloric acid and sulfuric acid.

19. The method of claim 13, wherein the step of eluting comprises washing the cation exchange resin with at least one salt and at least one acid.

20. The method of claim 19, wherein the wash solution comprises at least one of sodium chloride and potassium chloride and at least one of hydrochloric acid and sulfuric acid.

21. The method of claim 13, further comprising crystallizing glucosamine after elution from the cation exchange resin.

22. The method of claim 1, further comprising washing glucosamine bound to the cation exchange resin.

23. The method of claim 1, wherein the N-acetylglucosamine is present in a composition having a concentration of between about 10% and about 70% prior to contact with the cation exchange resin.

24. The method of claim 1, wherein the N-acetylglucosamine is present in a composition having a concentration of between about 20% and about 50% prior to contact with the cation exchange resin.

25. The method of claim 1, wherein the N-acetylglucosamine is contacted with the cation exchange resin for a period of between about 30 minutes and about four hours.

26. The method of claim 1, wherein the N-acetylglucosamine is contacted with the cation exchange resin in the presence of a gas.

27. The method of claim 26, wherein the gas is nitrogen.

28. The method of claim 1, wherein the N-acetylglucosamine is produced by a fermentation process.

29. The method of claim 28, wherein the N-acetylglucosamine is present in a fermentation medium.

30. The method of claim 29, wherein the fermentation medium has been partially purified.

31. The method of claim 1, wherein the glucosamine is recovered from the cation exchange resin and the yield of glucosamine from N-acetylglucosamine is at least about 50%.

32. The method of claim 1, wherein the glucosamine is recovered from the cation exchange resin and the yield of glucosamine from N-acetylglucosamine is at least about 70%.

33. A method of producing glucosamine comprising:
a. contacting N-acetylglucosamine with a cation exchange resin in the hydrogen form wherein an acetyl group is hydrolyzed to produce glucosamine;
b. washing the cation exchange resin with at least one water wash; and,
c. washing the cation exchange resin with an acid to elute a stable acid salt of glucosamine.

34. A method of producing stable salts of glucosamine comprising:
a. contacting N-acetylglucosamine with a cation exchange resin in the hydrogen form wherein an acetyl group is hydrolyzed to produce glucosamine;
b. washing the cation exchange resin with at least one water wash; and,
c. washing the cation exchange resin with a solution containing a salt selected from the group consisting of sodium chloride, potassium chloride, sodium sulfate, potassium sulfate, sodium bisulfate, potassium bisulfate and combinations thereof to elute a solution of glucosamine salts.

35. A method of producing glucosamine comprising:
a. culturing a microorganism in a fermentation medium wherein the microorganism produces N-acetylglucosamine;
b. deionizing the fermentation medium containing N-acetylglucosamine;
c. contacting the N-acetylglucosamine with a cation exchange resin in the hydrogen form wherein an acetyl group is hydrolyzed to produce glucosamine;
d. washing the cation exchange resin with at least one water wash;
e. washing the cation exchange resin with hydrochloric acid to elute glucosamine hydrochloride;
f. contacting the eluted glucosamine hydrochloride with an aqueous alcohol to crystallize the glucosamine hydrochloride; and,
g. drying the glucosamine hydrochloride crystals.

36. A method of producing salts of glucosamine comprising:
a. culturing a microorganism in a fermentation medium wherein the microorganism produces N-acetylglucosamine;
b. deionizing the fermentation medium containing N-acetylglucosamine;
c. contacting N-acetylglucosamine with a cation exchange resin in the hydrogen form wherein an acetyl group is hydrolyzed to produce glucosamine;
d. washing the cation exchange resin with at least one water wash;
e. washing the cation exchange resin with a solution containing a salt selected from the group consisting of sodium chloride, potassium chloride, sodium sulfate, potassium sulfate, sodium bisulfate, potassium bisulfate and combinations thereof to elute a solution of glucosamine salts;
f. contacting eluted glucosamine salts with an aqueous alcohol to crystallize the glucosamine salt; and,
g. drying the glucosamine salt crystals.

37. A method of producing salts of glucosamine comprising:
a. culturing a microorganism in a fermentation medium wherein the microorganism produces N-acetylglucosamine;
b. deionizing the fermentation medium containing N-acetylglucosamine;
c. contacting N-acetylglucosamine with a cation exchange resin in the hydrogen form wherein an acetyl group is hydrolyzed to produce glucosamine;
d. washing the cation exchange resin with at least one water wash;
e. washing the cation exchange resin with a solution containing at least one salt selected from the group consisting of sodium chloride, potassium chloride, sodium sulfate, potassium sulfate, sodium bisulfate, potassium bisulfate and combinations thereof, and at least one acid selected from the group consisting of hydrochloric acid, sulfuric acid and mixtures thereof to elute a solution of glucosamine salts;
f. contacting eluted glucosamine salts with an aqueous alcohol to crystallize the glucosamine salt; and,
g. drying the glucosamine salt crystals.
